(19) United States
(12) Patent Application Publication
(10) Pub. No.: US 2012/0116458 A1
(43) Pub. Date: May 10, 2012

(54) MODULAR PIVOTABLE SCREW ASSEMBLY AND METHOD

(75) Inventors: Matthew Van Nortwick, Memphis, TN (US); Dennis G. Crandall, Mesa, AZ (US)

(73) Assignee: WARSAW ORTHOPEDIC, INC., Warsaw, IN (US)

(21) Appl. No.: 12/941,671

(22) Filed: Nov. 8, 2010

(51) Int. Cl. A61B 17/88; A61B 17/86

(52) U.S. Cl. 606/279; 606/305

(57) ABSTRACT

A bone anchor assembly and a rigid extension rod removably coupleable thereto. The bone anchor assembly includes a screw base and a pivoting post. The post has a first recess in a proximal endface thereof, which may have first threads therein. The extension rod has a distal section separated from a proximal section by an intermediate section. The rod distal section has external threads that are threadingly engageable with the threads of the post recess. The rod third section may have external third threads of a different size. The rod intermediate section has an unthreaded exterior. When the extension rod is coupled to the post, an exterior of the second section of the extension rod is aligned with the exterior of the post. Other connection approaches may be used. An optional spacer may also be used to couple the rod to the post.

### BACKGROUND

The present invention relates to a spinal implant and a manner of using the same, and more particularly, but not exclusively, relates to an orthopedic device for treatment of spinal deformities.

The use of spinal implants to address orthopedic injuries and ailments has become commonplace in spinal surgery. In this arena, it is often desired to decrease the invasiveness of the procedures, improve implant integrity, reduce the potential for revision surgery, and provide more positive patient outcomes. Many times, implants utilize bone anchors to position the implant relative to the spine or portions thereof. Examples of such bone anchors are pedicle screws and the like. In some situations, it may be desirable for the bone anchor to be adjustable such that a proximal portion of the bone anchor can pivot relative to the bone engaging portion of the bone anchor, so as to facilitate assembly of the implant and alignment of the vertebrae to which the implant is attached. However, the morphology of different spines of different patients, and between different portions of the spine of a single patient, tends to be quite variable. Accordingly, different length bone anchors may be needed in different situations. Providing such different lengths has proven difficult in practice. As such, there is a need for additional contributions in this area of technology.

### SUMMARY

According to one aspect, a bone anchor apparatus includes a bone anchor assembly and an extension rod. The bone anchor assembly includes a screw base and a post. The screw base has a head and a shaft. The shaft extends along a first central longitudinal axis from a distal end to a proximal end proximate the head. The shaft is positionable in bony structure to secure the screw base to the bony structure, and may include bone engaging threads. The post is rigid and extends along a second central longitudinal axis from a distal section to a proximal section. The post distal section is pivotally mounted at the head of the screw base so that the post is pivotable relative to the screw base to orient the second central longitudinal axis in a plurality of angular orientations relative to the first longitudinal axis. The post has a proximally facing first recess in a proximal endface thereof. The first recess has first threads therein. An exterior of the post is unthreaded. The elongate rigid extension rod is removably coupleable to the post. The extension rod has a distal first section separated from a proximal third section by an intermediate second section. The rod first section has second external threads that are threadingly engageable with the first threads of the post recess. The rod third section has external third threads of a size different than the first and second threads. The rod second section has an unthreaded exterior. When the extension rod is coupled to the post by engaging the first threads with the second threads, an exterior of the second section of the extension rod is aligned with the exterior of the post.

In various embodiments, the apparatus may further include a spacer. The spacer may have a distal fifth section and a proximal sixth section. The fifth section has fifth external threads that are threadingly engageable with the first threads. The sixth section has a proximally facing second recess in a proximal endface thereof substantially similar to the first recess of the post. The extension rod may be coupled to the post via the spacer.

### BRIEF DESCRIPTION OF THE DRAWINGS

FIG. 1 is a posterior view of a spinal fixation system according to one or more embodiments engaged to the spinal column of a patient.

FIG. 2 shows an extension rod mated to a bone anchoring assembly according to one or more embodiments.

FIG. 3 shows a partially exploded view of FIG. 2.

FIG. 4 shows the bone anchoring assembly of FIG. 2 with the post angled relative to the screw base.

FIG. 5 shows a partial cross-section of the bone anchoring assembly of FIG. 2.

FIG. 5A shows a cross-section of the post of FIG. 2

FIG. 5B shows a proximal end view of the post of FIG. 2.

FIG. 6 shows another embodiment wherein an extension rod is mated to a spacer, which is in turn mated to a bone anchoring assembly.

FIG. 7 shows a partially exploded view of FIG. 6.

FIG. 8 shows a cross-section of the spacer of FIG. 6.

### DESCRIPTION OF THE ILLUSTRATED EMBODIMENTS

The present invention provides for a modular pivoting bone screw assembly and related method. In one embodiment, the bone screw assembly 30 includes a screw base 32 with a post 50 pivotally mounted thereto. A recess in the proximal endface of the post 50 includes threads. An extension rod 70 is provided that is screwable into the recess. The extension rod 70 includes a distal threaded section separated from a proximal threaded section by an intervening unthreaded intermediate section. When screwed into the recess, the extension rod 70 is collinear with the post 50, and the exterior of the unthreaded intermediate section is aligned with the post 50's exterior. This allows a connector to be slid down the extension rod 70 and onto the post 50, where it may be used to secure a spinal rod. After securing the connector in place, the extension rod 70 may be removed by simply unscrewing the extension rod 70 from the post 50. In other embodiments, an additional spacer 90 is interposed between the post 50 and the extension rod 70 to provide additional length. Additional details and embodiments are discussed below.

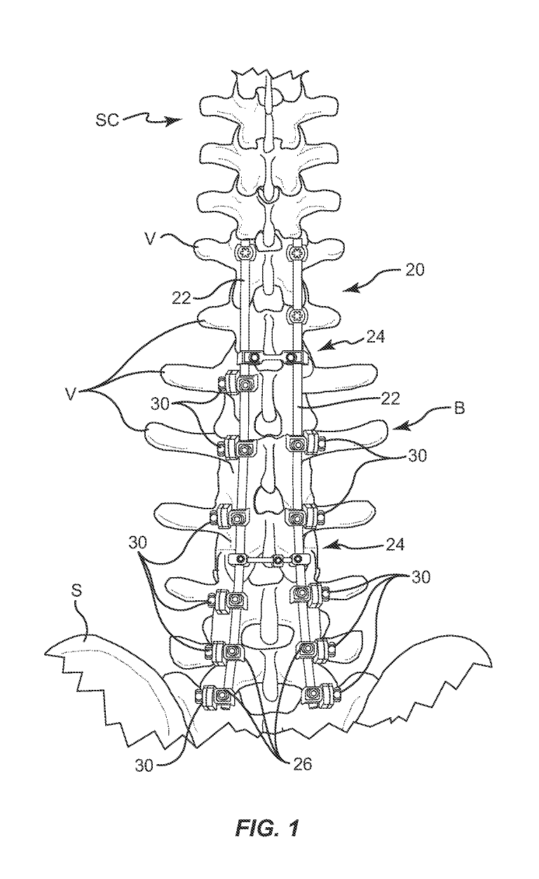

FIG. 1 illustrates an implanted posterior spinal stabilization system 20. More specifically, as depicted in FIG. 1, system 20 is affixed to one or more of bones B of the spinal column segment SC from a posterior approach. Bones B include one or more vertebrae V and sacrum S of spinal column segment SC. Spinal stabilization system 20 may be employed in spinal column segments SC including sacrum S and one or more vertebrae V, or in spinal column segments that comprise two or more vertebrae V. In system 20, bone anchors 30 are affixed to various locations of the spinal column segment SC and interconnected with spinal rods 22, which are positioned on opposite sides of the medial or sagittal plane of the spinal column and extend in the cephalad-caudal direction to provide bi-lateral stabilization. Spinal rods 22 may also be interconnected by one or more crosslink devices 24 that extend medially-laterally across the sagittal plane to provide additional stabilization for treating spinal disorders.

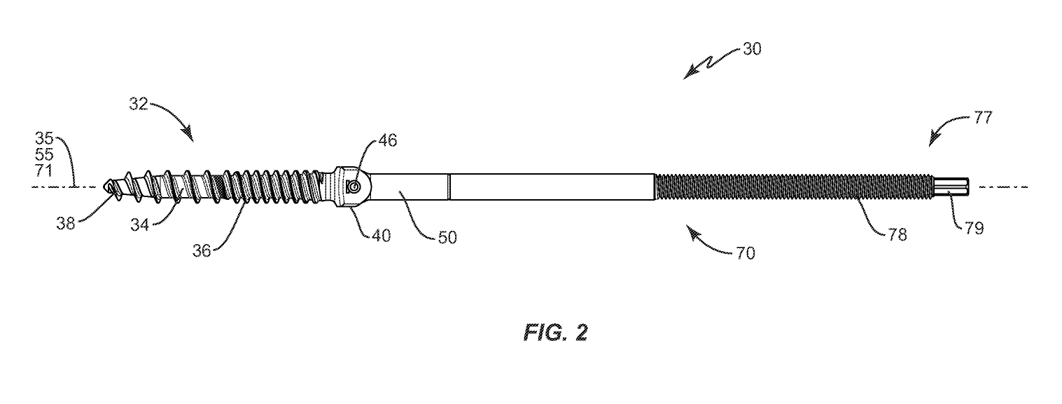

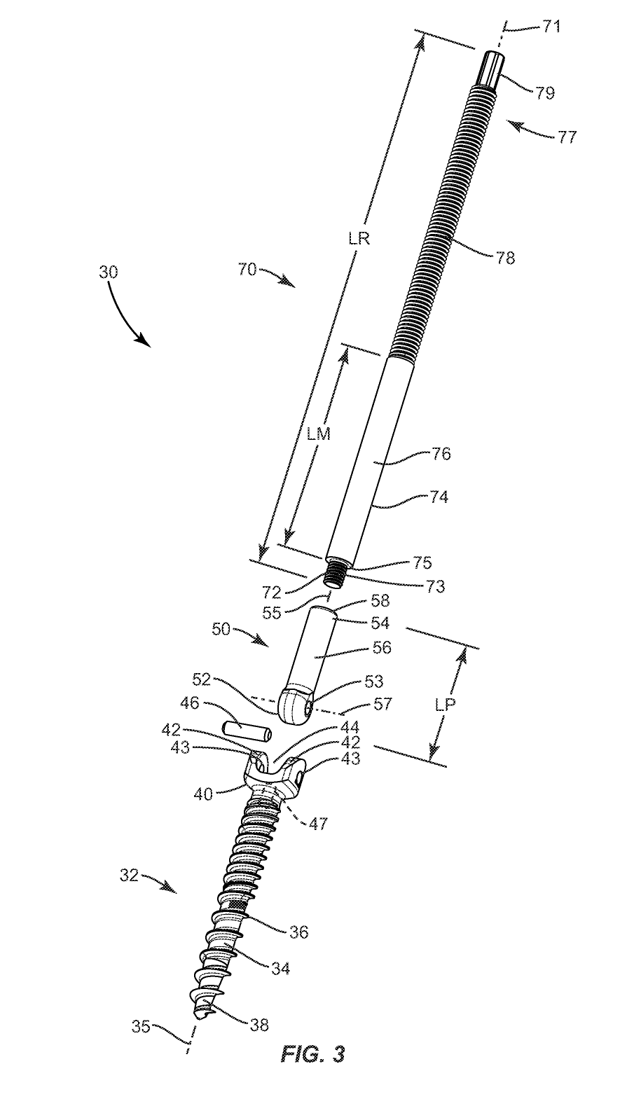

Posterior stabilization system 20 may be used for, but is not limited to, treatment of degenerative spondylolisthesis, fracture, dislocation, scoliosis, kyphosis, spinal tumor, and/or a failed previous fusion associated with spinal column segment SC. Furthermore, spinal column segment SC may comprise any one or combination of the cervical, thoracic, lumbar and sacral regions of the spinal column. In certain procedures, spinal stabilization system 20 is secured to a spinal column segment SC with bone anchors 30 that include a distal bone engaging portion 32 and a post 50 that extends proximally from the bone engaging portion 32 (see FIG. 2-3). The spinal rods 22 are offset to a side of respective ones of the bone anchors 30 and mounted to the post 50 with a suitable connector 26. In one specific embodiment, the connector 26 is like that found in Medtronic Sofamor Danek's TSRH® 3D spinal system. Of course, any suitable connector may be used to secure spinal rods 22 to one or more of the bone anchors 30. Furthermore, the bone anchors 30 may be employed in surgical procedures and fixation systems like those described in the 2009 Medtronic Sofamor Danek USA, Inc. publication entitled “TSRH-3D PLUS MPA™ SPINAL INSTRUMENTATION DEFORMITY AND DEGENERATIVE SURGICAL TECHNIQUE”, which is incorporated herein by reference.

The spinal rods 22 may be solid or hollow along some or all of its length and/or may be of homogenous or heterogeneous composition. The spinal rods 22 may also be of uniform cross-section along its entire length, or have a variable cross-section along its length. The spinal rods 22 may include one or more interconnected spinal rod portions that lengthen or adjust in length to accommodate growth of spinal column segment SC over time in the cephalad-caudal directions. The spinal rods 22 can be rigid, flexible, or include one or more flexible portions to permit at least limited spinal motion.

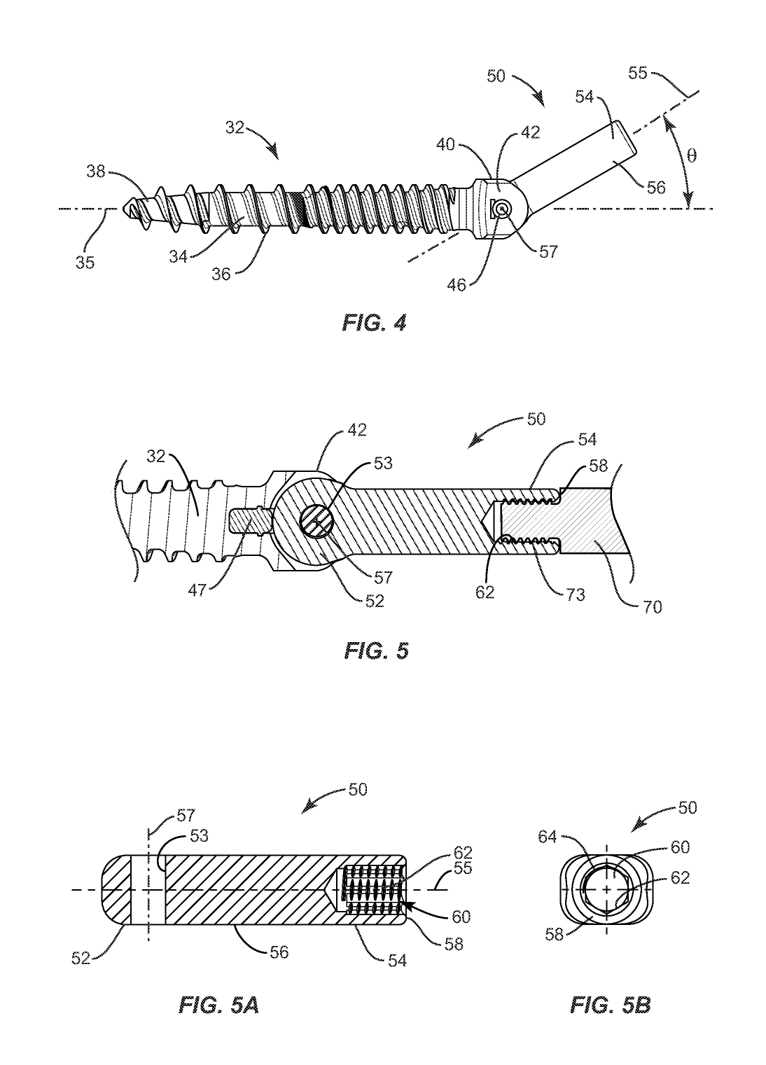

Referring to FIGS. 2-5B, one embodiment of a bone anchor assembly 30 is shown with an associated extension rod 70. The bone anchor assembly 30 includes a screw base 32 and a post 50 pivotally mounted to the screw base 32. The screw base 32 includes a shaft 34 and a head 40. The shaft 34 extends along a longitudinal axis 35 from the head 40 to a distal tip 38. The shaft 34 advantageously includes bone engaging threads 36 thereon. These threads 36 may be of any bone engaging type known in the art. For example, the threads 36 may be one continuous bone-cutting thread, or the shaft may include two or more longitudinal sections that have differing thread patterns. The distal tip 38 may advantageously have a pointed configuration to facilitate entry into bone. The head 40 advantageously includes two spaced apart arms 42 that form a yoke or recess 44 therebetween. The arms 42 extend proximally away from the shaft 34, while being slightly laterally offset from the longitudinal axis 35. The arms 42 may include suitable holes 43 for accepting a corresponding pivot pin 46 or pivot pins, as discussed further below. In some embodiments, a friction insert 47 may be disposed in the head 40 so as to partially extend into the recess 44.

A post 50 is pivotally mounted to the head 40 of the screw base 32. The post 50 is elongate along longitudinal axis 55 from a distal mounting section 52 to a generally cylindrical proximal section 54. The mounting section 52 may have a rounded outer surface, with a centrally located transverse bore 53 for accepting the pivot pin 46 so as to pivotally mount post 50 to screw base 32. The post proximal section 54 advantageously has an unthreaded exterior surface 56, which is generally smooth. The proximal endface 58 of the post 50 includes a longitudinally extending hole or recess 60. This recess 60 has threads 62 of a first female thread type, e.g., an M3.5 x. 06 thread form. The recess 60 is for matingly receiving the extension rod 70, as discussed further below. The length of the post 50 is indicated at LP. The recess 60 may optionally also have a faceted cross-section, such as with a plurality of mutually opposing facets 64, for example, hexagonal. These facets 64 may assist in attaching devices other than extension rod 70, such as nitinol extensions, to the post 50.

The post 50 is pivotable relative to the screw base 32 about pivot axis 57. Thus, the post 50 may be disposed such that an angle Θ is formed between the longitudinal axis 35 of the screw base 32 and the longitudinal axis 55 of the post 50. The angle Θ, in some embodiments, may in the range of about +90° to about −90°, for a total pivot arc of about 180°. In other embodiments, the allowed pivoting angle Θ may be greater or lesser, and the allowed pivot arc may or may not be symmetric about screw base longitudinal axis 35. For example, the pivoting may be approximately 90° clockwise, but about 0° counter-clockwise. Further, the post 50 may be freely pivotable, or may be provided with some pivoting resistance. For example, friction insert 47 in head 40 may be soft and bear against the perimeter of post mounting section 52 in order to provide some resistance to relative movement. Further, the post 50 may or may not be lockable in a desired position, see co-pending application Ser. No. 12/851,714, filed 6 Aug. 2010, and entitled “Locking Mechanisms for Pivoting Bone Anchors,” the disclosure of which is incorporated herein by reference.

The extension rod 70 is a rigid elongate member that extends along rod longitudinal axis 71. See, for example, FIG. 3. The rod includes a distal section 72 and a proximal section 77, with an intermediate section 74 disposed therebetween. The distal section 72 includes external threads 73 that are complementary to threads 62 of post 50. See, for example, FIG. 5. Further, the length of rod distal section 72 is shorter than the “depth” of post recess 60. The distal section 72 abuts the intermediate section 74 at a shoulder 75. The intermediate section 74 has an unthreaded exterior 76, which is advantageously smooth and round in cross-section. The intermediate section 74 extends for a length LM. The proximal section 77 abuts the intermediate section 74 and includes exterior threads 78. These threads 78 are of a different configuration than threads 73 of distal section 72, in part because the threaded portion of proximal section 77 is larger in cross-section than distal section 72. The proximal section 77 may also include a drive portion 79 that allows the extension rod 70 to be tightened into the post 50. For example, the drive portion 79 may have a faceted cross-sectional profile, such as hexagonal, and may be smaller in size than the threaded portion of proximal section 77. The overall length of the extension rod 70 is LR. Advantageously the extension rod length LR is longer than post length LP.

The bone anchor assembly 30 and extension rod 70 may be used to secure a spinal rod 22 in place. In one method, the bone anchor assembly 30 is mounted to a vertebra V by screwing shaft 34 into the corresponding bone B. The shaft 34 should be screwed in sufficiently to firmly anchor the bone anchor assembly 30 to the vertebra V and such that the pivot plane of the post 50 is aligned as desired. The extension rod 70 is then screwed into the post 50 by inserting the rod distal section 72 into the post recess 60 and inter-engaging threads 72 and threads 62, such as by using a suitable tool and drive portion 79 to turn extension rod 70 relative to post 50. The extension rod 70 should be screwed in completely, such that the shoulder 75 marking the transition from the rod distal section 72 to the intermediate section 74 is abutting the proximal endface 58 of post 50. This mounting of the extension rod 70 to the post 50 results in the extension rod axis 71 being collinear with the post axis 55. In addition, the exterior surface 76 of the rod intermediate section 74 is aligned with exterior surface 56 of post 50. At this point, the combination of the post 50 and extension rod 70 remains pivotable relative to the screw base 32. A connector 26 is then placed over the extension rod 70 and slid downward toward post 50. A spinal rod 22 is joined to the connector 26, and suitable instrumentation is used to force the connector 26 further toward, and then onto, post 50, advantageously using threads 78 of rod proximal section 77. When connector 26 is positioned as desired on post 50, connector 26 is locked into position on the post 50 in a conventional fashion. In some embodiments, the post 50 is then locked relative to the screw base 32 to prevent further pivoting. The extension rod 70 is then disengaged from the post 50, e.g., distal section 72 is unscrewed from post recess 60. Note that disengaging the extension rod 70 from post 50 shortens the effective length of post 50 without having to cut or otherwise alter post 50. The surgical procedure then continues as appropriate and the surgical site is closed in a conventional fashion.

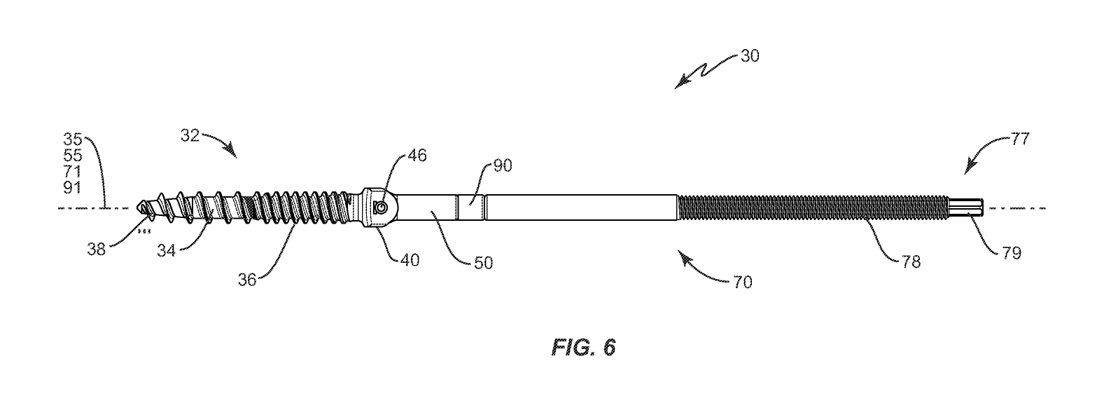

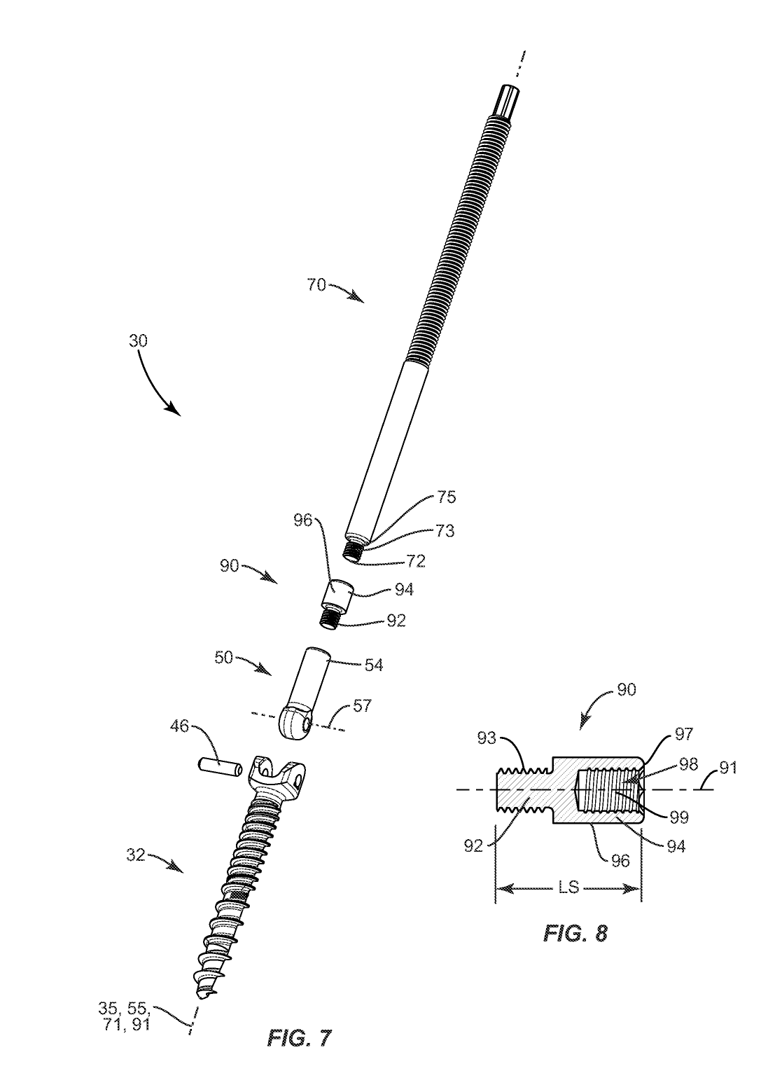

In some embodiments, an additional spacer 90 may be employed. One embodiment of a spacer 90 is shown in FIGS. 6-8. The spacer 90 extends along spacer longitudinal axis 91 and includes a distal section 92 and a proximal section 94. The distal section 92, which is advantageously short and therefore may be referred to as stub section 92, has external threads 93 thereon that are advantageously of the same configuration as threads 73 on rod distal section 72. Indeed, the spacer distal section 92 is advantageously substantially identical to the rod distal section 72. The spacer 90 also includes a generally cylindrical proximal section 94. The proximal section 94 advantageously has an unthreaded exterior surface 96, which is advantageously smooth and mimics the exterior 76 of rod intermediate section 74, albeit advantageously in a shorter length. The proximal endface 97 of spacer 90 includes a longitudinally extending hole or recess 98 that is substantially identical to post recess 60, with threads 99. The overall length of spacer 90 is LS. Advantageously the spacer length LS is shorter than post length LP.

The spacer 90 may be disposed between the post 50 and the extension rod 70 and used to couple one to the other. One method of using the spacer 90 involves placing the bone anchoring assembly 30 as generally described above. If it is determined that additional effective height is needed for post 50, the spacer 90 is mounted to the post 50 by engaging threads 93 with post recess threads 62, either before or after implantation of the screw base 32. The extension rod 70 is coupled to the post 50 via the spacer 90 by engaging threads 73 with spacer recess threads 99. The connector 26 is slid from extension rod 70 to spacer 90, toward post 50. The connector 26 may be mounted to the post 50 as described above, in which case both the extension rod 70 and the spacer 90 should be decoupled from the post 50 before closing the surgical site. Alternatively, the connector 26 may be mounted to both the post 50 and the spacer 90, in which case the spacer 90 may be left in place (but the extension rod 70 removed).

The addition of the extension rod 70, and the optional spacer 90, allows a single size of bone anchor assembly 30 to be used in a variety of situations. Depending on where in the spine the device is to be located, and the patient's morphology, either the bone anchor assembly 30 or the bone anchor assembly 30 and the spacer 90 may be implanted during the surgical procedure. Further, the length provided by the extension rod 70 (and the optional spacer 90) allows the spinal rod to be anchored via the bone anchoring assembly 30 even in situations where the patient's spinal geometry differs significantly from the desired geometry. And, the reversible mounting approach to coupling the extension rod 70 to the post 50 allows the extension rod 70 to be decoupled from the screw base 32 (and post 50) during the surgical procedure without the need to cut the extension rod 70; thus, the surgical procedure is simplified.

In the discussion above, it has been assumed that male threads 73, 93 and female threads 62, 99 are used to couple together post 50, extension rod 70, and spacer 90. While threaded connections are believed advantageous, such is not required in all embodiments. In some embodiments other form of reversible male/female connections may be used in one or more of the couplings. For example, reversible snap lock connections, reversible wedge lock connections; eccentric cam lock connections, reversible bayonet-type connections, and the like could be used. Thus, threaded connections are considered to be just one example of male/female connection mechanisms, i.e. reversible connections where one part fits within another part so as to couple the two parts together for joint movement.

In the discussion above, the screw base 32 was discussed in the context of having an integrally formed shaft 34 and head 40. However, in other embodiments, the head 40 may be a separate component that is attached to the shaft 34. In any event, the head 40 provides a connection that allows the post 50 to pivot relative to the shaft 34.

The bone anchor assemblies 30, extension rods 70, and spacers 90 discussed herein can be provided in a number of sizes and configurations, and kits thereof, including varying lengths, diameters and bone screw thread arrangements. The post 50 can include a smooth outer surface 56, and may also include a tool engaging configuration formed on its inner and/or outer surfaces. The bone anchor assemblies 30 discussed herein can be formed of titanium, stainless steel, cobalt-chrome or any other suitable biocompatible metal or non-metal material.

While spinal rods 22 have been used as illustrative connecting elements 22, in other embodiments the connecting elements 22 may be any suitable spinal stabilization element positionable along the spinal column, including plates, bars, tethers, wires, cables, cords, inflatable devices, expandable devices, and formed in place devices, for example. Further, while the method(s) described above have been discussed in the context of implantation within a living patient for the treatment of various spinal disorders, such is not required. Indeed, the various apparatus and methods described herein may also be used in a non-living situation, such as within a cadaver, model, and the like. The non-living situation may be for one or more of testing, training, and demonstration purposes.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A bone anchor apparatus, comprising:
a bone anchor assembly comprising:
a screw base having a head and a shaft; the shaft extending along a first central longitudinal axis from a distal end to a proximal end proximate the head; the shaft being positionable in bony structure to secure the screw base to the bony structure;
a rigid post extending along a second central longitudinal axis from a distal section to a proximal section; the distal section being pivotally mounted at the head of the screw base so that the post is pivotable relative to the screw base to orient the second central longitudinal axis in a plurality of angular orientations relative to the first longitudinal axis; the orientations lying in a plane disposed parallel to the first longitudinal axis;
the post comprising a proximally facing first recess in a proximal endface thereof; the first recess having first threads therein; an exterior of the post being unthreaded;
an elongate rigid extension rod removably coupleable to the post; the extension rod having a distal first section separated from a proximal third section by an intermediate second section; the distal first section having second external threads that are threadingly engageable with the first threads; the third section of the rod having external third threads of a size different than the first and second threads; the second section having an unthreaded exterior;
wherein, when the extension rod is coupled to the post by engaging the first threads with the second threads, an exterior of the second section of the extension rod is aligned with the exterior of the post.

2. The bone anchor apparatus of claim 1 wherein the first recess of the post comprises a plurality of facet sections disposed in opposing relation to each other.

3. The bone anchor apparatus of claim 2 wherein the first recess of the post comprises six facet sections disposed in a hexagonal pattern.

4. The bone anchor apparatus of claim 1 wherein the distal section of the post further comprises a bore disposed transverse to the second axis; further comprising one or more pins extending into the bore and pivotally mounting the post to the screw base.

5. The bone anchor apparatus of claim 1 wherein the shaft comprises bone engaging threads thereon.

6. The bone anchor apparatus of claim 1 wherein the post is shorter in length than the intermediate section of the rod.

7. The bone anchor apparatus of claim 1 wherein the extension rod further comprises a faceted drive section disposed proximally relative to threads of the third section of the rod.

8. The bone anchor apparatus of claim 1:
further comprising a spacer having a distal fifth section and a proximal sixth section;
the fifth section having fifth external threads that are threadingly engageable with the first threads;
the sixth section comprising a proximally facing second threaded recess in a proximal endface thereof; the second threaded recess having sixth threads therein that are threadably engageable with the second threads.

9. The bone anchor apparatus of claim 8 wherein the extension rod is coupled to the post via the spacer.

10. The bone anchor apparatus of claim 8 wherein the spacer is removably coupled to the post via engagement of the fifth treads and the first threads.

11. A bone anchor apparatus, comprising:
a bone anchor assembly comprising:
a screw base having a head and a shaft; the shaft extending along a first central longitudinal axis and being positionable in bony structure to secure the screw base to the bony structure;
a rigid post extending along a second central longitudinal axis from a distal section to a proximal section; the distal section pivotally mounted at the head of the screw base so that the post is pivotable relative to the screw base to orient the second axis in a plurality of angular orientations relative to the first axis;
the post comprising a proximally facing first recess in a proximal endface thereof; the first threaded recess having a female portion of a first type male/female connection mechanism; an exterior of the post proximal section being unthreaded;
an elongate rigid extension rod having a distal first section separated from a proximal third section by an intermediate second section; the first section having a male portion of the first type of male/female connection mechanism; the third section having an external advancement structure thereon; the second section having an exterior that is unthreaded and of a different cross-sectional profile than both the first and third sections;
wherein, when the extension rod is removably coupled to the post by engaging the male and female portions of the first type male/female connection mechanism, an exterior of the second section of the extension rod is aligned with the exterior of the post.

12. The bone anchor apparatus of claim 11:
further comprising a spacer having a distal fifth section and a proximal sixth section; an exterior of the sixth section being unthreaded;
the fifth section having a male portion of the first type male/female connection mechanism;
the sixth section comprising a proximally facing second recess in a proximal endface thereof; the second recess having a female portion of the first type male/female connection mechanism.

13. The bone anchor apparatus of claim 12 wherein the spacer is longitudinally shorter than the post; wherein the rod is coupled to the post via the spacer; wherein the post, the spacer, and the rod are collinear.

14. The bone anchor apparatus of claim 11 wherein the post is pivotally coupled to the screw base so as to be able to rotate relative thereto at least 90 degrees, but less than 180 degrees.

15. The bone anchor apparatus of claim 11 wherein the head defines a receptacle that receives said post therein.

16. A surgical method, comprising:
providing a bone screw assembly having a rigid post pivotally coupled to a screw base; the screw base having external bone engaging threads thereon; the post having a proximally facing first threaded recess in a proximal endface thereof; the first threaded recess having first threads therein; an exterior of the post being unthreaded;
coupling the bone screw assembly to a bony structure by engaging the bone engaging threads therewith;
coupling a rigid extension rod to the post in collinear fashion; the rod having a first section disposed towards the post, a third section disposed away from the post, and a second section disposed between the first and third sections; the second section being unthreaded; the first section having external threads that are threadingly engagable with the first threads; the third section having external threads thereon larger than the first threads;
thereafter, mounting a spinal rod to the bone screw assembly via a connector by moving the connector over the extension rod toward and onto the post and thereafter securing the connector to the post;
thereafter, disengaging the extension rod from the post.

17. The surgical method of claim 16:
further comprising, prior to said mounting, disposing a spacer longitudinally between
the post and the first section; the spacer having a second threaded recess and a threaded stub; the disposing comprising engaging the stub with the threads of the first recess and engaging the first section of the rod with the second recess;
wherein the mounting comprises moving the connector over the spacer toward and onto the post;
further comprising; after the mounting, disengaging the spacer from the post.

18. The surgical method of claim 16 wherein said coupling the rigid extension rod to the post comprises inter-engaging the threads of the first section with the first recess of the post.

19. The surgical method of claim 16 further comprising thereafter closing the surgical site without shortening the post.
